(19) United States
(12) Patent Application Publication
(10) Pub. No.: US 2023/0343207 A1
(43) Pub. Date: Oct. 26, 2023

(54) METHOD FOR DETECTING A CONFLICT

(71) Applicant: Siemens Healthcare GmbH, Erlangen (DE)

(72) Inventors: Marcus PFISTER, Bubenreuth (DE); Christian KAETHNER, Freiburg (DE)

(73) Assignee: Siemens Healthcare GmbH, Erlangen (DE)

(21) Appl. No.: 18/305,768

(22) Filed: Apr. 24, 2023

(30) Foreign Application Priority Data
Apr. 26, 2022 (DE) 10-2022-204002.3
May 20, 2022 (DE) 10-2022-202047.9

(51) Int. Cl. G08C 25/00; G16H 40/63; G16H 40/67; A61B 34/35

(52) U.S. Cl. CPC G08C 25/00; G16H 40/63; G16H 40/67; A61B 34/35; G06V 40/174

(57) ABSTRACT

The invention relates to a computer-implemented method for detecting a conflict between a local control means and a distantly disposed remote control means. The method comprises a method step of receiving a first signal from the local control means. The method comprises a further method step of receiving a second signal from the remote control means. The method comprises a further method step of checking whether a conflict is present based on the first and the second signal. If a conflict was detected during the check, the method comprises a method step of providing conflict information, wherein the conflict information comprises information that a conflict is present.

### CROSS-REFERENCE TO RELATED APPLICATION(S)

The present application claims priority under 35 U.S.C. § 119 to German Patent Application No. 10 2022 204 002.3, filed Apr. 26, 2022 and German Patent Application No. 10 2022 205 047.9, filed May 20, 2022, the entire contents of each of which are incorporated herein by reference.

### FIELD

One or more example embodiments of the present invention relates to a method for detecting a conflict between a local control means and a remote control means disposed at a distance, and to a system which is embodied to carry out the method.

### RELATED ART

It is known that an intervention, in particular a medical intervention, can be performed jointly by staff deployed on site, or local staff, and staff disposed at a distance. The medical intervention is in this case performed on a patient. The medical intervention can in particular relate to a treatment and/or a diagnosis. The medical intervention may involve an insertion of a catheter and/or stent, for example.

The local staff are typically positioned in this case close to the patient. The remote staff are meanwhile typically disposed at a distance. “At a distance” in the present context means for example in a different room, in a different building, in a different city, in a different country or on a different continent, etc.

In this context, the intervention typically comprises a plurality of operating steps. The operating steps typically build one on top of the other. In other words, the individual operating steps are typically dependent on one another. An individual operating step can in this case be performed either by the local staff or by the remote staff. Typically, a fixed assignment exists dictating who performs which operating step. Effective coordination between the local and the remote staff is essential here.

In particular, the intervention can be performed by means of a robotic system or robot. In particular, at least one operating step can be performed by the robotic system. The robotic system can in this case be controlled or operated by the remote staff. The local staff are embodied to perform additional operating steps directly on the patient or on the robotic system. In particular, these additional steps can be performed on the instruction of the remote staff. For example, the local staff can fit the robotic system with a medical instrument or object by means of which a next operating step of the intervention can be performed based on control of the robotic system by the remote staff.

It is necessary that no conflicts that may have consequences for the patient arise between the local staff and the remote staff during the intervention. In other words, it is essential for a successful performance of the intervention that the local staff and the remote staff act in a well-coordinated manner when performing the various interdependent operating steps.

To avoid a conflict, it is known to indicate step by step to each member of staff which operating step or which action is to be performed next. Alternatively or in addition, it is known to block the robot for a remote control action by the remote staff until for example the local staff has finished setting up the robotic system and the next operating step can be performed by remote control of the robotic system. Regulatory actions of this kind governing the intervention or the control of the intervention are typically already implemented as a preventive measure.

### SUMMARY

However, both the step-by-step indicating of actions and the time-dependent blocking of the robotic system are time-consuming and can lead to a delay in the intervention.

One or more example embodiments of the present invention provide a method which permits an intervention to be performed as time-efficiently as possible while at the same time avoiding conflict.

The object may be achieved by means of a method for detecting a conflict between a local control means and a distantly disposed remote control means, by means of a system for detecting a conflict between a local control means and a distantly disposed remote control means, by means of a computer program product, and by means of a computer-readable storage medium according to the independent claims. Advantageous developments are set forth in the dependent claims and in the following description.

### BRIEF DESCRIPTION OF THE DRAWINGS

Characteristics, features and advantages of will become clearer and more readily understandable in connection with the following figures and their descriptions. At the same time, the figures and descriptions are not intended to limit the invention and its embodiments in any way.

Like components are labeled with corresponding reference signs in different figures. The figures are generally not to scale.

In the figures:

FIG. 1 shows a first exemplary embodiment of a method for detecting a conflict between a local control means and a distantly disposed remote control means,

FIG. 2 shows a second exemplary embodiment of a method for detecting a conflict between a local control means and a distantly disposed remote control means,

FIG. 3 shows a third exemplary embodiment of a method for detecting a conflict between a local control means and a distantly disposed remote control means,

FIG. 4 shows a fourth exemplary embodiment of a method for detecting a conflict between a local control means and a distantly disposed remote control means, and

FIG. 5 shows a conflict detection system for identifying a conflict between a local control means and a distantly disposed remote control means.

### DETAILED DESCRIPTION

Inventive achievements of the object are described below both in relation to the claimed devices or systems and in relation to the claimed method. Features, advantages or alternative embodiments mentioned in this context are equally to be applied also to the other claimed subject matters, and vice versa. In other words, the object-related claims (which are directed for example to a device) can also be developed by means of the features that are described or claimed in connection with a method. The corresponding functional features of the method are in this case embodied by means of corresponding object-related modules.

One or more example embodiments of the present invention relates to a computer-implemented method for detecting a conflict between a local control means and a distantly disposed remote control means. The method comprises a method step of receiving a first signal from the local control means. The method further comprises a method step of receiving a second signal from the remote control means. The method also comprises a method step of checking, on the basis of the first and the second signal, whether a conflict is present. If a conflict was detected during the check, the method additionally comprises a method step of providing conflict information. In this case the conflict information comprises information indicating that a conflict is present.

The local control means and the remote control means are embodied to jointly carry out or perform a process or an intervention. In particular, the local control means and the remote control means are embodied to perform a medical intervention. The medical intervention can in this case be performed on a patient. The medical intervention can in this case be in particular a minimally invasive examination or a minimally invasive intervention. For example, the medical intervention can be an insertion of a stent or a catheter examination, etc. The intervention can in particular comprise a plurality of operating steps. The individual operating steps can in this case be interdependent, in particular build on top of one another.

The remote control means comprises a computational unit. The remote control means can be connected with a medical device and/or a local display via a data transmission line. The medical device can be used to carry out a medical intervention and is placed at the patient/the place where the intervention takes place. The medical device can for example be a robot or a robotic system. The local display can be a monitor where some instructions from the remote staff can be displayed for the local staff. In particular the remote control means can comprise a user interface. The user interface is adapted to receive a user input from the remote staff. E.g., the user interface can be a touchscreen, a computer mouse, a computer keyboard, a voice control means etc. The user input can be “translated” by the computational unit in order to use the medical device or to display anything on the local display. In particular, the remote control means can comprise a camera and/or a microphone which is adapted to monitor the remote control staff.

The local control means can in particular be operated by local staff. Alternatively or in addition, the local control means can monitor the local staff for example visually or acoustically. The local staff can in particular be medical staff. The local staff is in particular disposed in the same room as the patient. Advantageously, the local staff is disposed close to the patient, i.e. in direct contact with the patient. The local staff can comprise one or more individuals.

The local control means comprises a computational unit. The computational unit can be adapted to translate a user input from the local staff into an instruction which can be used to carry out the instruction of the local staff. The instructions can be configured to carry out a medical intervention. In particular, the local control means can comprise a user interface which can receive a user input from the local staff. E.g., the user interface can be a touchscreen, a computer mouse, a computer keyboard, a voice control means etc. The user input can be translated by the computational unit to make a medical device to carry out the medical intervention. The medical device can be used to carry out a medical intervention and is placed at the patient/the place where the intervention takes place. The medical device can for example be a robot or a robotic system. In particular, the local control means can comprise a camera or a microphone to monitor the local staff.

The remote control means is disposed at a distance or remotely from the local control means. In other words, the remote control means is disposed in a different room or in a different building or in a different city or in a different country from the local control means. The remote control means can in particular be operated by remote staff. Alternatively or in addition, the remote control means can monitor remote staff for example visually or acoustically. The remote staff are in this case in particular medical staff. The remote staff can comprise one or more individuals.

The remote staff can in particular be in contact visually and/or acoustically with the local staff.

In embodiments of the invention, the remote control means can be embodied to control a robotic system or a robot. The robotic system is in this case embodied to perform the intervention.

In the method step of receiving the first signal, the first signal is received from the local control means. The first signal is in this case received by means of an interface. The first signal can in this case comprise information about the intervention. The first signal can in particular comprise a control signal for performing at least one operating step of the intervention. Alternatively or in addition, the first signal can comprise information about the local staff. In this case the information about the local staff can in particular comprise information about a level of satisfaction of the local staff and/or about a mood of the local staff. In particular, the first signal can comprise the information about the local staff indirectly, for example in the form of a video and/or audio recording of the local staff, from which the information about the local staff can be derived.

In the method step of receiving the second signal, the second signal is received from the remote control means. The second signal is in this case received by means of the interface. The second signal can in this case comprise information about the intervention. The second signal can in particular comprise a control signal for performing at least one operating step of the intervention. In this case the second signal can be embodied to perform a different operating step to the first signal. Alternatively or in addition, the second signal can comprise information about the remote staff. The information about the remote staff can in this case comprise for example information about a level of satisfaction of the remote staff and/or a mood of the remote staff. In particular, the second signal can comprise the information about the remote staff indirectly, for example in the form of a video and/or audio recording of the remote staff, from which the information about the remote staff can be derived.

In the checking method step, it is checked on the basis of the first and the second signal whether a conflict is present. In other words, it is checked whether the first or the second signal or both signals in combination point to a conflict between the local control means and the remote control means. In particular, the combination of the first and the second signal is checked in order to establish whether a conflict is present.

A conflict may be present for example when the local control means performs an operating step which is at odds with an operating step performed or planned by the remote control means or vice versa. In other words, a conflict may be characterized by a contradiction between the first and the second signal.

A conflict may also be present when it is recognized, based on the information about the local and/or the remote staff, that at least the local or the remote staff are dissatisfied.

A conflict may also be present when the first or the second signal comprises a plurality of instructions which are contradictory to one another. Alternatively or in addition, a conflict may be present when the first or the second signal comprises a number of instructions given at short time intervals to one another. Alternatively or in addition, a conflict may be present when the first or the second signal comprises instructions which are not output in an order corresponding to the operating steps of the intervention.

The method step of providing conflict information is performed if it is recognized during the checking step that a conflict is present. The conflict information can for example indicate the presence of a conflict. The conflict information can be displayed to the local and/or the remote staff on a screen or monitor, for example. Alternatively or in addition, the conflict information can be embodied to control a warning light which lights up when a conflict is present.

The inventors have recognized that a conflict can be detected by evaluating the first signal and the second signal from the local control means and the remote control means, respectively. The inventors have recognized that there is no need to intervene in the process or become involved in the intervention as long as no conflict is detected. In this way, for example, already well-drilled teams (local and remote staff) can carry out the intervention without interruption. The inventors have recognized that it is possible to intervene in the process or intervention when a conflict is detected. Such an intervention can be for example providing the conflict information about the presence of a conflict situation.

According to one or more example embodiments of the present invention, the first and/or the second signal can be provided by means of at least one of the following inputs: a user input, an audio input, a video input, a system input.

In this case the provided input can be received by the local control means or the remote control means. The local control means or the remote control means then provides the first or second signal for receiving.

The user input can in particular be provided by means of a user interface. The user interface can be embodied in this case to receive a user input from the local or remote staff. The user input can in particular be a control signal. The control signal is in this case embodied to perform a next operating step. For example, the user input can be a request from the remote staff to the local staff to carry out a particular operating step or to carry out an operating step in a particular manner. For example, the user input by the remote staff can state “Insert the catheter”.

The audio input can in particular be provided by means of a microphone. The microphone is in particular embodied to receive voices from the local or remote staff. In particular, the audio input can comprise a communication between the local and the remote staff when the first and the second signal form a common audio input. Alternatively, the audio input may comprise only one side of the communication. In particular, the audio input can comprise instructions or requests from the remote staff to the local staff.

The video input can in particular be provided by means of an optical camera. The camera can in this case be embodied to film the local or the remote staff.

The system input can in particular be provided by a system which is embodied to carry out the intervention. The system input can be for example a command to the robotic system that is to perform the intervention. In this case the system input can be initiated by means of a user input. For example, a user input by the remote staff can initiate a system input to the robotic system to perform an operating step. Alternatively, the system input can be for example an error message etc. of the robotic system.

The inventors have recognized that different signals can be used for detecting a conflict. The inventors have recognized that these inputs or signals can be actively provided by the local and/or the remote staff. The inventors have recognized that these inputs or signals can be provided alternatively or in addition for example by continuous monitoring of the local and/or remote staff.

According to one or more example embodiments of the present invention, the first and/or the second signal comprise/comprises a video input. In this case the checking method step comprises a facial recognition and/or an object recognition function.

In other words, the checking method step comprises facial recognition and/or object recognition when the first and/or the second signal comprise/comprises a video input. The facial recognition and/or the object recognition are/is applied in this case to the corresponding signal which comprises the video input.

The facial recognition can in particular be embodied to determine a level of satisfaction or mood or frame of mind of the staff imaged in the video input. In particular, the level of satisfaction or mood or frame of mind of the local and/or remote staff can be determined in this way.

The object recognition is embodied to identify an object or an article or an instrument that is used or inserted by the local or remote staff.

Known algorithms can be used for the facial recognition and/or object recognition.

The inventors have recognized that it can be identified on the basis of the facial recognition whether the local and/or the remote staff are satisfied or irritated. Irritation can in this case indicate a conflict. The inventors have also recognized that the object recognition can be used to check whether an operating step is performed correctly. For example, by means of the object recognition it can be checked in a video input included in the first signal whether a user input included in the second signal has been performed correctly. If the user input of the remote staff for example stipulates the insertion of a particular catheter, it can be checked by means of the object recognition of the video input whether the required catheter has been inserted by the local staff.

According to one or more example embodiments of the present invention, the first and/or the second signal comprise/comprises an audio input. In this case the checking method step comprises a voice recognition function.

In other words, the checking method step comprises voice recognition if the first and/or the second signal comprise/comprises an audio input. The voice recognition can then be applied to the audio input.

The voice recognition can in particular be embodied to determine a stress level in a voice captured in the audio input. In other words, the voice recognition can be embodied to determine a level of satisfaction or mood or frame of mind in the voice captured in the audio input. The voice can in this case be a voice of a member of the local or the remote staff.

Alternatively or in addition, the voice recognition can be embodied to determine words in the audio input. In particular, the voice recognition can be embodied to detect a multiple repetition of the same words.

Alternatively or in addition, the voice recognition can be embodied to detect a contradiction in a spoken instruction. For example, the voice recognition can detect if the local or the remote staff give contradictory instructions. Furthermore, the voice recognition can detect if different instructions are given in quick succession or in a confused manner. This too can indicate a conflict.

The voice recognition can be based on a known algorithm.

The inventors have recognized that it can be identified on the basis of the voice recognition in combination with the object recognition whether a spoken command or request is implemented correctly. The inventors have also recognized that it can be detected, based on the voice recognition applied to the audio input, whether a voice captured in the audio input is stressed or expressing dissatisfaction. Stress or dissatisfaction can in this case indicate a conflict. The inventors have furthermore recognized that a multiple repetition of the same words or the same sense of words can also indicate a conflict. For example, the remote staff can request the local staff to perform a particular operating step by means of an audio input. If the local staff does not perform the operating step or performs it incorrectly, the remote staff will repeat the request several times. This can be a pointer to a conflict.

According to one or more example embodiments of the present invention, the first signal comprises a first point in time and the second signal a second point in time. In this case the first point in time indicates when the first signal was generated. In this case the second point in time indicates when the second signal was generated. In this case the checking method step takes into account a chronological sequence of the first and the second point in time.

During the check it can be taken into account in this case in particular whether a time interval between the first and the second point in time exceeds or falls short of a predetermined time interval. For this purpose, a lower and an upper limit value can be specified in advance for the time interval. Falling short of the lower limit value and exceeding the upper limit value can indicate a conflict. In this case the lower and the upper limit value can be dependent on a current operating step of the intervention. In other words, the lower and the upper limit value can be dependent on when the first and the second signal are provided during the intervention.

The inventors have recognized that in this way it can be detected whether a response to a request for example takes a particularly long time. By means of the second signal at the second point in time, for example, the remote staff can request the local staff to perform a particular operating step. The first signal can then indicate by means of the first point in time when the operating step was then actually performed by the local staff. If an unexpectedly long time interval between such a second and such a first signal can be detected, this can indicate a conflict. The inventors have also recognized that a very short time interval, i.e. for example an overlapping of the first and the second signal, can also indicate a conflict. In this case one staff possibly do not wait until the other staff have completed their operating step, which is captured by the corresponding signal.

According to one or more example embodiments of the present invention, the method also comprises the following method steps if a conflict has been detected: determining a de-escalation signal as a function of the conflict and providing the de-escalation signal. In this case the de-escalation signal is embodied to resolve the conflict.

The de-escalation signal can in particular be provided to the local staff and/or the remote staff. Alternatively or in addition, the de-escalation signal can be provided to a system used to perform the intervention, for example the robotic system. The de-escalation signal is embodied to resolve or remove the conflict. The de-escalation signal can for example draw the attention of the local and/or the remote staff to the conflict. In addition, the de-escalation signal can provide a suggestion for resolving the conflict. The de-escalation signal is therefore dependent on the conflict or on the type of conflict. Alternatively, the de-escalation signal can intervene in the intervention. For example, the de-escalation signal can block an access to the robotic system for the remote staff at least temporarily. Alternatively, the de-escalation signal can comprise clear guidelines for the local and/or the remote staff. These guidelines can specify precisely how and when which staff is to perform which operating step.

In the method step of determining the de-escalation signal, the de-escalation signal suitable for resolving the conflict is determined. In particular, the de-escalation signal is determined by means of a computing unit. In other words, the computing unit is embodied to determine the de-escalation signal. In this case the determined de-escalation signal is dependent on the previously determined conflict. In other words, the de-escalation signal is dependent on the type of or reason for the conflict.

In the method step of providing the de-escalation signal, the de-escalation signal is provided in such a way that it can resolve the conflict. In this case the de-escalation signal is provided by means of the interface. In other words, the interface is embodied to provide the de-escalation signal. For example, the de-escalation signal can be provided visually and/or acoustically to the local and/or the remote staff.

When provided visually, the de-escalation signal can be provided on a screen or monitor. In this case the de-escalation signal can for example comprise an image and/or a pictogram and/or a text and/or a film sequence, etc.

When provided acoustically, the de-escalation signal can be provided by means of a loudspeaker. In this case the de-escalation signal can be for example a warning sound or an announcement, etc.

Alternatively, the de-escalation signal can be provided to a system used for performing the intervention, for example to a robotic system. The system can in this case receive the de-escalation signal by means of an interface. The de-escalation signal can in this case be embodied to inhibit or block the system, at least temporarily.

The inventors have recognized that in the event of a conflict, the conflict can be efficiently resolved by means of the de-escalation signal. In this case the de-escalation signal is provided only when a conflict is present. In this way an unnecessary corrective adjustment of the intervention can be avoided. In this way it can be ensured that a time-intensive corrective adjustment is performed only when necessary, i.e. when a conflict is present, whereas otherwise the intervention can be performed as time-efficiently as possible.

According to an optional aspect, the de-escalation signal is provided until a confirmation is received by the local staff and/or by the remote staff or until the conflict has been resolved or until a current operating step has been completed or for the duration of a predetermined time period.

The de-escalation signal can therefore be provided in particular until such time as the local staff and/or the remote staff have confirmed the de-escalation signal. The confirmation can be issued by means of a user input. The user input can be submitted in particular by means of a user interface, for example a touch-sensitive screen (touchscreen) or a keyboard or a touchpad or a computer mouse or a voice input. When confirming, the local and/or the remote staff indicate that they have understood the de-escalation signal. In particular, the local and/or the remote staff can indicate that they have responded to the de-escalation signal.

Alternatively, the de-escalation signal can continue to be provided until it can be identified on the basis of a new first and a new second signal that the conflict has been resolved. In other words, the first and the second signal are received once again and evaluated or checked as described above. If the conflict is still present, the de-escalation signal continues to be provided. If the conflict is no longer present, the de-escalation signal is no longer provided.

Alternatively, the de-escalation signal can continue to be provided until a current operating step in which the conflict has been detected has been terminated or completed. In particular, the de-escalation signal can continue to be provided until the current operating step has been successfully completed.

Alternatively, the de-escalation signal can be provided for the duration of a predetermined time period.

The inventors have recognized that in most cases the de-escalation signal needs to be provided for a limited time only. The inventors have recognized that a new check to establish whether a conflict is present can be conducted for this purpose. The inventors have recognized that as an alternative the de-escalation signal can be terminated or removed by the staff themselves. The inventors have recognized that more personal responsibility is conferred on the staff in this way. The inventors have recognized that the de-escalation signal can be provided for different lengths of time or as a function of different conditions depending on the severity or frequency of a conflict. The inventors have recognized that in the case of a serious or frequently occurring conflict, for example, the de-escalation signal can continue to be provided until it is identified that no conflict is present any longer. In contrast thereto, for example in the case of a minor or one-off conflict, the de-escalation signal can continue to be provided until a confirmation by the local and/or the remote staff is received or for the duration of a predetermined time period.

According to one or more example embodiments of the present invention, the de-escalation signal triggers a blocking of the local control means or the remote control means.

In other words, the de-escalation signal is embodied to block control actions by the local staff or control actions by the remote staff. For example, a blocking of remote control can mean that the remote staff are no longer able to control the robotic system remotely. Alternatively or in addition, a blocking can mean that the local staff are no longer hearing acoustic instructions from the remote staff and/or vice versa.

The inventors have recognized that the blocking of local control or remote control is an efficient means of regulating the performance of the intervention. The inventors have recognized that in this way conflicts due to control inconsistences can be resolved. The inventors have recognized that in this way it can be laid down which staff are to make a decision concerning the intervention by blocking the control by the other staff.

According to one or more example embodiments of the present invention, if a conflict has been detected, the method also comprises a method step of determining a reason for the conflict based on the check. In this case the de-escalation signal comprises information about a reason for the conflict.

The method step of determining a reason for the conflict is in this case performed in particular by means of the computing unit. In other words, the computing unit is embodied to determine the reason for the conflict.

The reason for the conflict is determined in this case in particular based on the checking method step. In particular, the reason for the conflict is determined based on the first and the second signal. In other words, the reason for the conflict can be derived from the first and the second signal.

In particular, the reason for the conflict can indicate why a conflict has been detected on the basis of the first and the second signal.

For example, the reason for the conflict can be an insertion of an incorrect object by the local staff. In this case the first signal can comprise a video input on which it is identified by means of object recognition which object is being inserted by the local staff. The second signal can then comprise a user input by the remote staff in which it is indicated which object is to be inserted. If an inconsistency is detected here, this is determined as the reason for the conflict.

The de-escalation signal can then comprise the reason for the conflict and inform the local or remote staff about the reason for the conflict. Alternatively or in addition, the de-escalation signal can be dependent on the reason for the conflict. In this case the de-escalation signal is embodied in such a way that it is suitable for removing the reason for the conflict.

The inventors have recognized that a knowledge of the reason for the conflict can help the local and/or the remote staff to understand how it came to the conflict. The inventors have recognized that the reason for the conflict can be taken into account in the determination of the de-escalation signal.

According to one or more example embodiments of the present invention, the de-escalation signal comprises an audio output and/or a video output. In this case the audio output and/or the video output are/is dependent on the reason for the conflict.

The de-escalation signal can be provided to the local and/or the remote staff acoustically or visually by means of the audio output or the video output, respectively.

The audio output can in this case be provided by means of a loudspeaker. The audio output can be a warning sound or an announcement, etc.

The video output can be provided by means of a screen or monitor. The video output can in particular comprise an illustration or an image or a pictogram or a film sequence or a text.

When the de-escalation signal is being determined, in particular the reason for the conflict is taken into account. The de-escalation signal can in this case in particular indicate what is the reason for the conflict and how the latter is to be resolved.

For example, it can be determined as a reason for the conflict that the local staff are not using the device requested by the remote staff. The de-escalation signal can then display an image of the right device to the local staff for example by video output and/or alert the local staff acoustically by audio output that the wrong device has been used.

The inventors have recognized that an audio and/or a video output are/is particularly suitable for providing the de-escalation signal to the local and/or the remote staff. The inventors have recognized that in this case various ways of conflict resolution can be employed.

According to one or more example embodiments of the present invention, if a conflict has been detected, the method also comprises a method step of determining a frequency of an occurrence of a conflict having the determined reason. In this case the de-escalation signal is dependent on the frequency.

The method step of determining the frequency is performed in this case by means of the computing unit. In other words, the computing unit is embodied to determine the frequency. The frequency indicates how often a conflict having the same reason has already been identified during the intervention. The frequency is therefore dependent on the reason for the conflict. The frequency can therefore increase during the intervention.

When the de-escalation signal is determined, the determined frequency is taken into account here. In this case, as the frequency increases, the de-escalation signal can intervene more and more in the intervention or correctively adjust the intervention more and more robustly.

The inventors have recognized that a more frequent occurrence of the same conflict, i.e. a conflict having the same reason, is attributable to a more fundamental problem. In other words, a frequent occurrence of the same conflict typically cannot be based only on a misunderstanding. The inventors have recognized that the de-escalation signal must then be adapted and must intervene in the intervention in a more radically corrective manner in order to avoid a repeat occurrence of the conflict. The inventors have recognized that as a result of the adaptive adjustment of the de-escalation signal, a distinction can be made between occasionally occurring conflicts and between more persistent conflicts. The inventors have recognized that in this way the intensity of the regulation of the intervention can be geared to whether the conflict is simply a misunderstanding or a more serious conflict between the local and the remote staff. In the case of a more serious conflict, a loss of time during the performance of the intervention must be accepted due to a more radical regulation in order to avoid further conflicts, whereas in the case of an isolated misunderstanding, mostly only a suggestion and assistance are necessary to resolve the misunderstanding or the conflict and no further loss of time needs to be conceded as a result of a corrective action.

According to one or more example embodiments of the present invention, the first signal comprises a first point in time and the second signal a second point in time. The first point in time in this case indicates when the first signal was generated. The second point in time indicates when the second signal was generated. In this case the first and the second signal are generated in connection with an intervention. In this case the de-escalation signal is dependent on a progress of the intervention at the first and/or second point in time.

In other words, the progress of the intervention at the first or second point in time is taken into account during the determination of the de-escalation signal.

The intervention is embodied in this case as described above. In particular, the intervention can be time-critical in different ways at different points in time. For example, if the intervention is a medical intervention, preparations for a medical intervention at the beginning of the intervention are less time-critical than an invasive part of the intervention in which the patient is subject to invasive treatment. In other words, the progress of the intervention can indicate how time-critical the intervention is at the first and/or second point in time.

During the determination of the de-escalation signal, it can therefore be taken into account how time-critical the intervention is at the first and/or second point in time.

In particular, the de-escalation signal can have a stronger regulating effect if the intervention is time-critical at the first and/or second point in time in order to resolve a conflict as quickly as possible. A stronger regulation during the overall intervention can in fact lead to a time delay in the overall intervention. In order to resolve an individual conflict, however, a brief strong regulation can help save time in order to ensure that the conflict has definitely been resolved. If the intervention is less time-critical at the first and/or second point in time, the de-escalation signal may initially comprise only pointers for resolving the conflict and correctively adjust the intervention as little as possible.

The inventors have recognized that in order to achieve as efficient a conflict resolution as possible, the progress of the intervention can be taken into account at the time the conflict occurs, i.e. at the first and/or second point in time. In this way it can be taken into account whether the progress of the intervention permits a minimally corrective conflict resolution to be realized, or whether it is necessary to intervene in the intervention in a robustly corrective manner in order to ensure that no fresh conflict occurs.

According to one or more example embodiments of the present invention, the first and the second signal are received in the course of an intervention. Following completion of the intervention, the method also comprises a method step of receiving feedback from the local control means and/or the remote control means in the form of a user input. In this case the feedback comprises an evaluation of the de-escalation signal.

The interface is in this case embodied to receive the feedback.

The user input can in this case be provided in particular by the local staff via the local control means and/or by the remote staff via the remote control means.

The feedback can comprise the evaluation of the de-escalation signal for example in the form of grades or in the form of a scale embodied in a different way. Alternatively or in addition, the feedback can comprise a written evaluation of the de-escalation signal in the form of a text.

If more than one de-escalation signal has been provided during the intervention, the feedback can comprise a collective evaluation for all the provided de-escalation signals. Alternatively, the feedback can comprise an evaluation for each individual de-escalation signal.

In embodiments of the invention, the feedback can be used in order to adapt the de-escalation signal based on the evaluation in a repeated performance of the method. In other words, the evaluation can be taken into account in the determining of the de-escalation signal in a repeated performance of the method.

The inventors have recognized that feedback by the local and/or remote staff during the determination of the de-escalation signal can be taken into account in a repeated performance of the method. The inventors have recognized that in this way a false interpretation of conflicts and consequently a providing of unnecessary de-escalation signals or of too weakly or too strongly regulating de-escalation signals can be avoided.

One or more example embodiments of the present invention furthermore relates to a conflict detection system for identifying a conflict between a local control means and a distantly disposed remote control means. The conflict detection system comprises an interface and a computing unit. In this case the interface and the computing unit are embodied to perform the following method steps: receiving a first signal from the local control means, receiving a second signal from the remote control means, checking, on the basis of the first and the second signal, whether a conflict is present and, if a conflict has been detected during the check, providing information that a conflict is present.

Such a conflict detection system can in particular be embodied to perform the above-described method for identifying a conflict between a local control means and a distantly disposed remote control means and its aspects. The conflict detection system is embodied to perform this method and its aspects in that the interface and the computing unit are embodied to perform the corresponding method steps.

One or more example embodiments of the present invention also relates to a computer program product comprising a computer program, as well as to a computer-readable medium. A largely software-based implementation has the advantage that conflict detection systems already used previously in the prior art can also be easily upgraded by means of a software update in order to operate in the manner described. In addition to the computer program, such a computer program product may, where applicable, comprise additional constituent parts, such as e.g. a set of documentation, and/or additional components, as well as hardware components, such as e.g. hardware keys (dongles, etc.) to enable use of the software.

In particular, one or more example embodiments of the present invention also relates to a computer program product comprising a computer program which can be loaded directly into a memory of a conflict detection system and having program sections for performing all the steps of the above-described method for detecting a conflict between a local control means and a distantly disposed remote control means and its aspects when the program sections are executed by the conflict detection system.

In particular, one or more example embodiments of the present invention relates to a computer-readable storage medium on which program sections are stored that can be read and executed by a conflict detection system in order to perform all the steps of the above-described method for detecting a conflict between a local control means and a distantly disposed remote control means and its aspects when the program sections are executed by the conflict detection system.

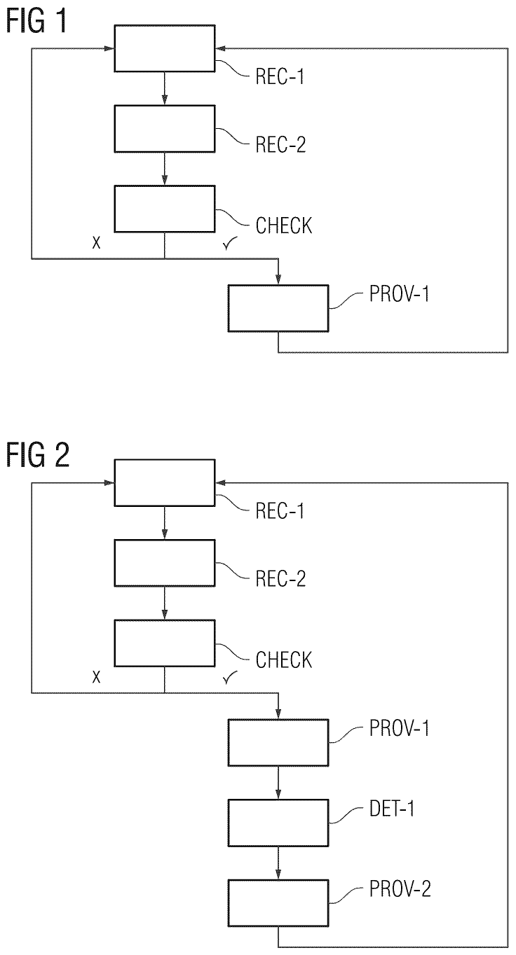

FIG. 1 shows a first exemplary embodiment of a method for detecting a conflict between a local control means and a distantly disposed remote control means.

The local control means and the remote control means are embodied to jointly perform an intervention, in particular a medical intervention. The local control means can be operated by local staff in this case. The remote control means can be operated by remote staff in this case.

Alternatively or in addition, the local control means is embodied for the visual and/or acoustic monitoring of the local staff. Alternatively or in addition, the remote control means is embodied for the visual and/or acoustic monitoring of the remote staff.

The intervention can comprise at least one operating step, in particular a plurality of operating steps. In this case each operating step is performed or initiated either by the local staff or by the remote staff. Typically, it is clearly defined who performs which operating step.

A medical intervention is in this case in particular an intervention performed on a patient. The intervention can in this case be an examination or an operation, etc. The intervention can be performed for example assisted by a robotic system. In this case one or more operating steps can be performed by means of the robotic system. In particular, the robotic system can be controlled in this case by the local or the remote staff.

During a medical intervention, the local staff are typically disposed or positioned directly by the patient or at least in direct proximity to the patient.

The remote staff is in this case disposed at a distance from the local staff. Accordingly, the remote control means is also disposed at a distance from the local control means. “At a distance” can in this context mean in a different room, in a different building, in a different city or in a different country, etc.

The local control means in this case provides a first signal. The first signal can comprise a control signal. The control signal is embodied to perform an operating step of the intervention. Alternatively or in addition, the first signal can comprise a monitoring signal. The monitoring signal is embodied to monitor the local staff visually or acoustically.

The remote control means in this case provides a second signal. The second signal can comprise a control signal. The control signal is embodied to perform an operating step of the intervention. Alternatively or in addition, the second signal can comprise a monitoring signal. The monitoring signal is embodied to monitor the remote staff visually or acoustically.

If the two signals comprise a control signal, the two control signals can be embodied in particular to perform two interrelated operating steps. If at least one of the signals comprises a monitoring signal, the corresponding signal has typically been captured at the point in time at which the other signal was captured. In other words, the two signals were then typically captured at least temporarily simultaneously.

In a method step of receiving REC-1 the first signal, the first signal provided by the local control means is received. In this case the first signal is received by means of an interface. In other words, the interface is embodied to receive the first signal.

In a method step of receiving REC-2 a second signal, the second signal provided by the remote control means is received. In this case the second signal is received by means of the interface. In other words, the interface is embodied to receive REC-2 the second signal.

In a checking method step CHECK, it is checked on the basis of the first and the second signal whether a conflict is present. In this case it is checked for example whether an inconsistency is present between the first and the second signal. Alternatively or in addition, it is checked whether a dissatisfaction of the local and/or the remote staff can be detected from one of the monitoring signals, which points to an inconsistency.

If a conflict has been detected, conflict information is provided in a providing method step PROV-1. The conflict information in this case comprises information that a conflict is present. In other words, the conflict information notifies the presence of the conflict.

The conflict information can in this case be provided in particular to the local staff and/or the remote staff. Alternatively, the conflict information can be provided to the local control means and/or the remote control means. Alternatively or in addition, the conflict information can be used for resolving the conflict. For this purpose, the conflict information can be provided to a system, in particular the local control means and/or the remote control means, which is embodied to resolve the conflict.

If no conflict has been detected, the intervention continues to be performed without interruption. In particular, it continues to be monitored in the course of the intervention whether a conflict occurs.

In an exemplary embodiment of the invention, the first and/or the second signal are/is provided by means of at least one of the following inputs: a user input, an audio input, a video input, a system input.

A user input is in this case actuated by the local staff and/or by the remote staff. The user input can in this case be entered by means of a user interface. The user input can in this case be embodied for example for communication between the local staff and the remote staff. For example, the user input can be a specification by the remote staff setting out how the local staff should perform a particular operating step of the intervention. The user input can in this case be in particular in written and/or acoustic form. In other words, the user input can be embodied as audio input and/or video input.

The audio input is in this case embodied for monitoring the local and/or the remote staff. In other words, the audio input can comprise a recording of the discussions of the local and/or the remote staff.

The video input is in this case embodied for monitoring the local and/or the remote staff. The video input can picture the local and/or the remote staff when performing the intervention.

The system input can be for example a command to a robotic system for performing at least one operating step of the intervention. The robotic system is in this case embodied to perform at least one operating step or to support the intervention. For example, the remote staff can control the robotic system by means of a system input when the local staff have prepared the intervention on site. In other words, the remote staff can initiate a system input for controlling the robotic system by means of a user input. Alternatively, the system input can be for example an error message of the local control means and/or the remote control means and/or the robotic system.

In embodiments of the invention, the checking method step CHECK comprises a facial recognition and/or an object recognition if the first and/or the second signal comprises a video input.

The facial recognition can be embodied to determine a frame of mind or a mood or a level of satisfaction of the staff pictured on the video input. In other words, the facial recognition is embodied to determine or detect a frame of mind expressed in faces pictured in the video input.

The object recognition is embodied in particular to identify an object or article relevant to the intervention in the video input. For example, it can be detected by means of the object recognition whether the local staff is using an object or article or instrument requested by the remote staff for performing an operating step of the intervention.

In embodiments of the invention, the first and/or second signal comprises an audio input. In this case the checking method step CHECK comprises voice recognition.

The voice recognition can be embodied to detect on the basis of the voice in the audio input whether the speaker is under stress or is angry. In other words, the voice recognition can be embodied to detect a frame of mind or mood or stress level of the speaker on the basis of the voice in the audio recording. The speaker can in this case be one or more individual members of the local and/or remote staff.

Alternatively or in addition, the voice recognition can be embodied to detect words in the audio input. In particular, it can be detected on the basis of the voice recognition whether certain words or a certain sense of words are/is repeated several times. This can indicate a conflict if a staff member repeats an instruction several times. Alternatively or in addition, an inconsistency in the words can also be detected. Thus, if the local or remote staff give two contradictory instructions in quick succession, this can be identified.

In embodiments of the invention, the first signal comprises a first point in time and the second signal a second point in time. In this case the first point in time indicates when the first signal was generated. In this case the second signal indicates when the second signal was generated. The checking method step CHECK takes into account a chronological sequence of the first and the second point in time.

If the first and the second signal are embodied for performing an operating step of the intervention in each case, it can be taken into account during the check CHECK for example whether both signals were generated in the right order in which the operating steps are also to be carried out. Alternatively or in addition, it can be checked during the check CHECK whether a time interval between the first and the second point in time is too long or too short. Too long or too short a time interval can indicate a conflict. For this purpose, an upper and/or a lower limit value for the time interval can be specified in advance. Falling short of the lower limit value can indicate too short a time interval. Exceeding the upper limit value can indicate too long a time interval.

FIG. 2 shows a second exemplary embodiment of a method for detecting a conflict between a local control means and a distantly disposed remote control means.

The method steps of receiving REC-1 a first signal, of receiving REC-2 a second signal, of checking CHECK and of providing PROV-1 the conflict information are embodied analogously to the description relating to FIG. 1. The examples in relation to the various embodiments can be applied to the following second exemplary embodiment.

In a further method step of determining DET-1 a de-escalation signal, the de-escalation signal is determined as a function of the conflict.

In a further method step of providing PROV-2 the de-escalation signal, the de-escalation signal is provided.

The de-escalation signal is embodied to resolve the conflict. In particular, the conflict can be resolved by providing PROV-2 the de-escalation signal. In particular, the de-escalation signal can be provided to the local and/or the remote staff. Alternatively or in addition, the de-escalation signal can be provided to the local control means and/or the remote control means. Alternatively or in addition, the de-escalation signal can be provided to the robotic system if it used for performing the intervention.

In particular, the de-escalation signal can be provided in such a way that the local and/or the remote staff are made aware of the conflict. Furthermore, by means of the de-escalation signal the local and/or the remote staff are provided with information on how the conflict can be resolved. For example, the de-escalation signal can be an image of an object which the remote staff would like to use and which the local staff is to insert or use on behalf of the remote staff.

In embodiments of the invention, the de-escalation signal triggers a blocking of the local control means or the remote control means. In particular, the de-escalation signal can in this case be embodied in such a way that the control means which is currently not to perform an operating step is blocked. For example, after an operating instruction is provided by the remote control means, the remote control means can be blocked in order to allow the local control means to perform the operating instruction without interference. In this way, for example, control of the robotic system by the remote staff can be blocked.

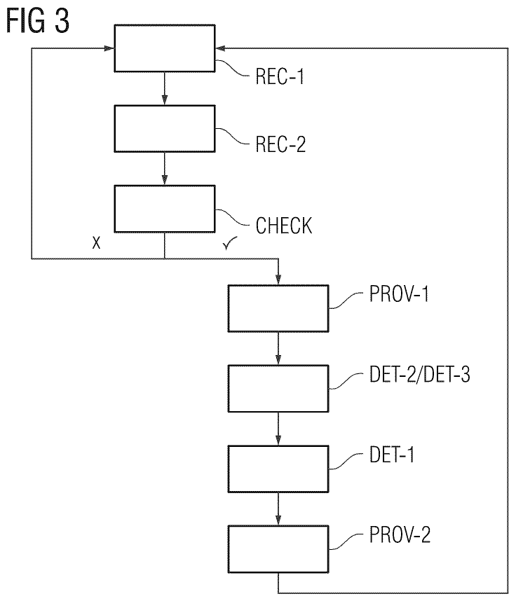

FIG. 3 shows a third exemplary embodiment of a method for detecting a conflict between a local control means and a distantly disposed remote control means.

The method steps of receiving REC-1 a first signal, of receiving REC-2 a second signal, of checking CHECK and of providing PROV-1 the conflict information are embodied analogously to the description relating to FIG. 1. The method steps of determining DET-1 a de-escalation signal and of providing PROV-2 a de-escalation signal are embodied analogously to the description relating to FIG. 2. The examples in relation to the various embodiments from the descriptions relating to FIGS. 1 and 2 can be applied to the following third exemplary embodiment.

In a method step of determining DET-2 a reason for the conflict, the reason for the conflict is determined based on the check CHECK. In this case the de-escalation signal comprises information about the reason determined for the conflict.

The reason for the conflict indicates what triggered the conflict. Alternatively or in addition, the reason for the conflict indicates why a conflict was identified or detected on the basis of the first and the second signal.

The de-escalation signal can then indicate the reason for the conflict. In addition, the de-escalation signal indicates, based on the reason for the conflict, how the latter is to be resolved. For example, the reason for the conflict can be an insertion, by the local staff, of an incorrect article or object or instrument not requested by the remote staff. The de-escalation signal can then indicate that the wrong object has been inserted and which object would be the right one.

In embodiments of the invention, the de-escalation signal comprises an audio output and/or a video output. The audio and/or the video output are/is dependent in this case on the reason for the conflict. In the above example, the video output can be a displaying of the correct or desired object. In the above example, the audio output can be a voice announcement stating which object is the desired or correct one. Alternatively or in addition, the audio output can be an output of a warning sound indicating when a conflict occurs.

Alternatively or in addition the method comprises a method step of determining DET-3 a frequency of an occurrence of a conflict having the determined reason. In this case the de-escalation signal is dependent on the frequency. In other words, in the method step of determining DET-3 the frequency, it is determined how frequently during the intervention a conflict having the same reason occurs. The de-escalation signal can then be dependent on how frequently during the process the conflict having the same reason as the currently detected conflict has already occurred. The more frequently the conflict has already occurred, the more strongly regulating can the de-escalation signal intervene in the intervention. When a conflict occurs for the first time in the intervention, for example, the de-escalation signal can be embodied in such a way that as a result of providing the de-escalation signal, the local and/or the remote staff are simply alerted to the conflict and the appropriate staff resolve the conflict themselves. If the same conflict occurs again, the de-escalation signal can comprise more information about the conflict, which information is provided to the appropriate staff. For example, the de-escalation signal can then comprise a video output which explains for which reason the conflict arose and how this can be resolved and avoided. If the same conflict occurs again, the de-escalation signal can comprise for example a blocking of the local control means or the remote control means.

In an embodiment of the invention, the first signal comprises a first point in time and the second signal a second point in time. As described above, the first point in time indicates when the first signal was generated and the second point in time indicates when the second signal was generated. In this case the first and the second signal are generated in connection with an intervention. In this case the de-escalation signal is dependent on a progress of the intervention at the first and/or second point in time.

The intervention is in this case embodied as described above. “In connection with the intervention” means that the first and the second signal are generated in the course of the intervention. The progress of the intervention can in this case indicate how time-critical the intervention is at the corresponding point in time. In a medical intervention, for example, the step of preparing the patient is typically not very time-critical. An invasive treatment of the patient, on the other hand, can in some cases be very time-critical. In other words, the intervention can comprise operating steps or phases which are more or less time-critical. In time-critical operating steps, the occurrence of a conflict necessitates a swift resolution of the conflict. Consequently, in such a case the de-escalation signal can intervene in a more strongly regulating manner in the intervention than in the case of a less time-critical operating step. Thus, depending on the progress of the intervention, the de-escalation signal can intervene in a more or less strongly corrective manner in the intervention. In a time-critical operating step, the de-escalation signal can strongly modulate the intervention, for example by clear operating instructions to the corresponding staff or by the above-described blocking of the local control means and/or the remote control means. Although a strong correction can subsequently lead to a delay in the overall intervention, a rapid and corrective intervention to resolve the acute conflict may nonetheless be necessary at a time-critical point in time. In the case of a less time-critical operating step, the de-escalation signal can for example simply draw the attention of the corresponding staff to the conflict and thus give the staff the opportunity to resolve the conflict themselves in an optimal manner.

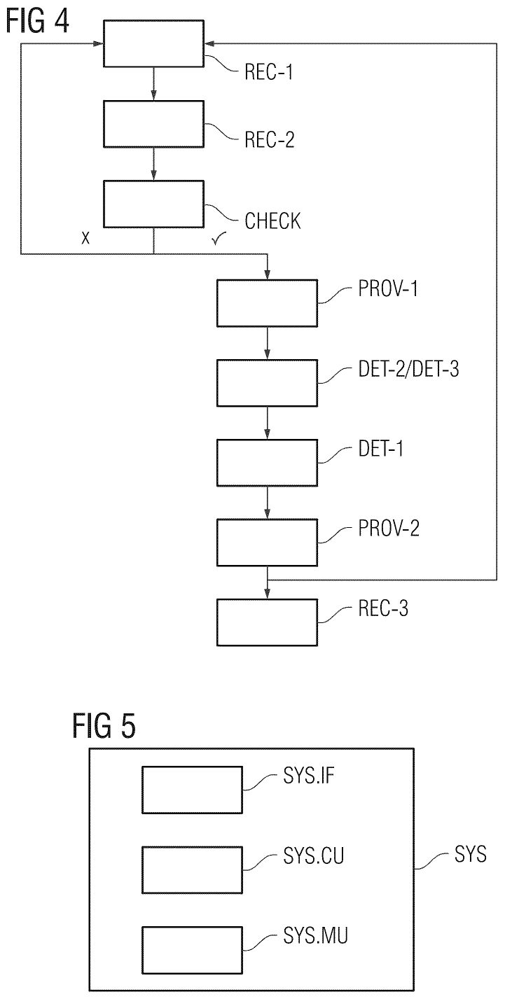

FIG. 4 shows a fourth exemplary embodiment of a method for detecting a conflict between a local control means and a distantly disposed remote control means.

The method steps of receiving REC-1 a first signal, of receiving REC-2 a second signal, of checking CHECK and of providing PROV-1 the conflict information are embodied analogously to the description relating to FIG. 1. The method steps of determining DET-1 a de-escalation signal and of providing PROV-2 a de-escalation signal are embodied analogously to the description relating to FIG. 2. The method steps of determining DET-2 a reason for the conflict and of determining DET-3 a frequency of the occurrence of a conflict having the determined reason are embodied according to the description relating to FIG. 3. The examples in relation to the various embodiments from the descriptions relating to FIGS. 1, 2 and 3 can be applied to the following fourth exemplary embodiment.

In a method step of receiving REC-3 feedback from the local control means and/or the remote control means, feedback is received in the form of a user input which comprises an evaluation of the de-escalation signal. In this case the feedback is received after completion of the intervention.

The feedback therefore indicates how satisfied a user, in particular a member of the local and/or the remote staff, was with the provided de-escalation signal during the intervention. If more than one de-escalation signal was provided during the intervention, the feedback can in particular comprise an evaluation of the totality of the de-escalation signals. Alternatively, the feedback can comprise an evaluation for every single one of the de-escalation signals. The evaluation can be expressed for example in the form of a scale having an arbitrary number of stages. For example, the evaluation can be expressed in the form of grades. Alternatively or in addition, the evaluation can comprise a written evaluation of the de-escalation signal in the form of a text.

If the method is repeated, the de-escalation signal can be adjusted based on the feedback.

FIG. 5 shows a conflict detection system SYS for identifying a conflict between a local control means and a distantly disposed remote control means.

The illustrated conflict detection system SYS for identifying a conflict between a local control means and a distantly disposed remote control means is embodied to perform an inventive method for detecting a conflict between a local control means and a distantly disposed remote control means. The conflict detection system SYS comprises an interface SYS.IF, a computing unit SYS.CU and a memory unit SYS.MU.

The conflict detection system SYS can in particular be a computer, a microcontroller or an integrated circuit (IC). Alternatively, the conflict detection system SYS can be a real or virtual computer network (a technical term for a real computer network is “cluster”; a technical term for a virtual computer network is “cloud”). The conflict detection system SYS can be embodied as a virtual system which is implemented on a computer or a real computer network or a virtual computer network (a technical term is “virtualization”).

The interface SYS.IF can be a hardware or software interface (for example a PCI bus, USB or Firewire). The computing unit SYS.CU can comprises hardware and/or software components, for example a microprocessor or a device known as an FPGA (Field Programmable Gate Array). The memory unit SYS.MU can be embodied as a volatile working memory (Random Access Memory or RAM) or as a nonvolatile mass storage device (hard disk drive, USB stick, SD card, solid state disk (SSD)).

The interface SYS.IF can in particular comprise a plurality of subsidiary interfaces that perform different method steps of the respective inventive method. In other words, the interface SYS.IF can be embodied as a plurality of interfaces SYS.IF. The computing unit SYS.CU can in particular comprise a plurality of subsidiary computing units that perform different method steps of the respective inventive method. In other words, the computing unit SYS.CU can be embodied as a plurality of computing units SYS.CU.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’, ‘interface’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing system or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Where not yet explicitly realized, though beneficial and within the spirit of the invention, individual exemplary embodiments and individual subordinate aspects or features thereof may be combined with one another or interchanged without leaving the scope of the present invention. Advantages of the invention that are described with reference to one exemplary embodiment are also relevant, where applicable, to other exemplary embodiments without being cited explicitly.

1. A computer-implemented method for detecting a conflict between a local control means and a distantly disposed remote control means, the method comprising:
receiving a first signal from the local control means;
receiving a second signal from the remote control means;
checking whether a conflict is present based on the first and the second signal; and
providing conflict information based on the checking, wherein the conflict information comprises information that a conflict is present.

2. The method of claim 1, wherein at least one of
the receiving the first signal receives the first signal via at least one of a user input, an audio input, a video input, or a system input; or
the receiving receives the second signal receives the second signal via at least one of the user input, the audio input, the video input, or the system input.

3. The method of claim 1, wherein
at least one of the first signal or the second signal comprises a video input, and
the checking checks using facial recognition or object recognition.

4. The method of claim 1, wherein
at least one of the first signal or the second signal comprises an audio input, and
the checking checks using voice recognition.

5. The method of claim 1, wherein
the first signal comprises a first time, the first time indicates when the first signal was generated,
the second signal comprises a second time, the second time indicates when the second signal was generated, and
the checking is based on a chronological sequence of the first and the second time.

6. The method of claim 1, further comprising:
determining a de-escalation signal as a function of the conflict; and
providing the de-escalation signal for resolving the conflict.

7. The method of claim 6, wherein the providing provides the de-escalation signal until a confirmation is received, until the conflict is resolved, until a current operating step is completed or for a predetermined time period.

8. The method of claim 6, wherein the de-escalation signal triggers a blocking of the local control means or the remote control means.

9. The method of claim 6, further comprising:
determining a reason for the conflict based on the checking, wherein the de-escalation signal comprises information about a reason for the conflict.

10. The method of claim 9, wherein
the de-escalation signal comprises at least one of an audio output or a video output, and
the at least one of the audio output or the video output is based on the conflict.

11. The method of claim 9, further comprising:
determining a frequency of an occurrence of the conflict having the determined reason, wherein the de-escalation signal is dependent on the frequency.

12. The method of claim 6, wherein
the first signal comprises the first time, the first time indicates when the first signal was generated,
the second signal comprises a second time, the second time indicates when the second signal was generated,
the first and the second signal are generated in connection with an intervention, and
the de-escalation signal is based on a progress of the intervention at at least one of the first time or the second time.

13. The method of claim 6, wherein the receiving the first signal and the receiving the second signal occur during an intervention, the method further comprises:
receiving feedback from at least one of the local control means or the remote control means via a user input, wherein the feedback comprises an evaluation of the de-escalation signal.

14. A conflict detection system for identifying a conflict between a local control means and a distantly disposed remote control means, the system comprising:
an interface; and
a computing unit, wherein the interface and the computing unit are configured to cooperatively perform,
receiving a first signal from the local control means,
receiving a second signal from the remote control means,
checking whether a conflict is present based on the first signal and the second signal, and
providing conflict information based on the checking, the conflict information including information that a conflict is present.

15. A non-transitory computer program product comprising a computer program having program sections, when executed by a conflict detection system, cause the conflict detection system to perform the method of claim 1.

16. A non-transitory computer-readable storage medium storing program sections, when executed by a conflict detection system, cause the conflict detection system to perform the method of claim 1.

17. The method of claim 5, further comprising:
determining a de-escalation signal as a function of the conflict; and
providing the de-escalation signal for resolving the conflict.

18. The method of claim 17, further comprising:
determining a reason for the conflict based on the checking, wherein the de-escalation signal comprises information about a reason for the conflict.

19. The method of claim 10, further comprising:
determining a frequency of an occurrence of the conflict having the determined reason, wherein the de-escalation signal is dependent on the frequency.

20. The method of claim 11, wherein
the first signal comprises the first time, the first time indicates when the first signal was generated,
the second signal comprises a second time, the second time indicates when the second signal was generated,
the first and the second signal are generated in connection with an intervention, and
the de-escalation signal is based on a progress of the intervention at at least one of the first time or the second time.
